(19) United States
(12) Patent Application Publication
(10) Pub. No.: US 2026/0219193 A1
(43) Pub. Date: Jul. 30, 2026

(54) Device and method for detecting molecules

(71) Applicant: FONDAZIONE ISTITUTO ITALIANO DI TECNOLOGIA, Genova GE (IT)

(72) Inventors: Francesco DE ANGELIS, Genova GE (IT); Francesco TANTUSSI, Genova GE (IT); Marzia IAROSSI, Genova GE (IT); Aliaksandr HUBAREVICH, Genova GE (IT); Angeia Federica DE FAZIO, Genova GE (IT); Devin Brent O'NEILL, Genova GE (IT)

(21) Appl. No.: 19/147,240

(22) PCT Filed: Jan. 16, 2024
(86) PCT No.: PCT/IB2024/050397

§ 371 Date: Jul. 10, 2025

(30) Foreign Application Priority Data
Jan. 17, 2023 (IT) 102023000000573

(51) Int. Cl. G01N 21/65

(52) U.S. Cl. CPC G01N 21/658

(57) ABSTRACT

A device for detecting molecules comprises a substrate with a first face and a second face, opposite each other, and at least one through channel with a first opening at the first face and a second opening at the second face. The channel is delimited by a side wall extending between the first and the second face. The device comprises a receptacle configured to contain a saline solution and the molecule to be detected. The substrate is insertable in the receptacle to be immersed in the saline solution during use. The device comprises a light source configured to illuminate the channel with a monochromatic electromagnetic radiation such that a hotspot is generated within the channel, and a generator configured to generate a potential difference between the first face and the second face of the substrate such that the molecule to be detected in the channel is moved during use. The device comprises a particle at least in part inserted in the channel and the generable hotspot is located between the particle and the side wall of the channel. The particle and the side wall are spaced so that the molecule to be detected flows between the particle and the side wall at the hotspot.

### TECHNICAL FIELD

The present invention relates to a device and a method for detecting molecules, in particular of proteins and nucleic acids. The present invention finds useful use in detecting and sequencing proteins. In particular, the present invention is applicable in the field of personalised medicine.

### DESCRIPTION OF THE PRIOR ART

Devices and methods for detecting and sequencing molecules, in particular DNA and RNA, are known in the state of the art. Such devices comprise a substrate of a plasmonic material, e.g., gold or silver. Such a substrate has a funnel-shaped channel, with a maximum opening and a minimum opening. The substrates thus described are called plasmonic nanopores, known in the state of the art.

In detail, in the devices of the prior art, the substrate comprises two opposite faces, subjected to a potential difference. Such a potential difference is configured to move, by means of electrophoresis, the molecule to be detected within the channel.

The devices of the prior art comprise a light source, configured to illuminate the channel with a monochromatic electromagnetic radiation with an appropriate wavelength. An intense electric field can thereby be generated within the channel, defined as a plasmonic hotspot. In particular, the plasmonic hotspot is generated at the minimum opening of the channel.

As the molecule to be detected passes within the channel, the hotspot stimulates such a molecule at a certain instant in time. At such an instant in time, the molecule emits a Raman signal, configured to generate a Raman spectrum.

In particular, in the case of proteins, it is possible to identify each amino acid when it is stimulated in the hotspot, at a certain instant in time, since each amino acid is recognisable thanks to the Raman spectrum thereof. Once the amino acid sequence has been identified, at least in part, it is possible to identify the protein present within the channel.

An example of a method for detecting and sequencing molecules, in particular a single amino acid in a protein, is shown in the document ‘Label-Free Optical Analysis of Biomolecules in Solid-State Nanopores: Toward Single-Molecule Protein Sequencing, ZHAO YINGQI et al.,’ according to which the molecules to be detected are absorbed by a particle retained in a channel.

US 2004/110208 A1 instead shows a device and method for detecting nucleotides, according to which nanoparticles are positioned in a channel and allow the molecules to be detected to pass between the particles.

### Problem of the Prior Art

Disadvantageously, in the devices and methods of the prior art, it is not always possible to generate a single hotspot. In fact, due to the geometry of the substrate, multiple hotspots may form. In this case, the quality of detection, and consequently of sequencing, is compromised, as each portion of the molecule is detected more than once.

Disadvantageously, in the devices and methods of the prior art, the hotspots generated are not sufficiently intense to detect single amino acids, single nucleotides, or low molecular weight molecules.

Disadvantageously, in the devices and methods of the prior art, the channel is too large to function effectively.

Disadvantageously, the devices of the prior art are more complicated to produce and thus less suitable for large-scale production.

### SUMMARY OF THE INVENTION

In this context, the technical task underlying the present invention is to provide a device and a method for detecting molecules which obviate the drawbacks of the prior art as described above.

In particular, it is the object of the present invention to provide a device and a method for detecting molecules in which a more intense and more localised hotspot can be generated.

It is still the object of the present invention to provide a device and a method capable of detecting single amino acids, single nucleotides or low molecular weight molecules.

Furthermore, it is a further object of the present invention to provide a device and a method for generating smaller channels.

Furthermore, it is a further object of the present invention to provide a device and a method for detecting molecules with an easier structure to produce.

The defined technical task and the specified objects are substantially achieved by a device and by a method for detecting molecules comprising the technical characteristics set forth in one or more of the appended claims.

Advantageously, the hotspot generated by such a device is more intense because, by inserting a particle within the channel, the hotspot is concentrated between such particle and a side wall of the channel.

Advantageously, it is possible to generate smaller channels, since the effective size can be adjusted by inserting a particle of a suitable size in the channel.

Advantageously, due to the higher hotspot strength, single amino acids, single nucleotides or low molecular weight molecules can be detected.

Advantageously, it is possible to increase the strength of the generable hotspot by adjusting the distance between the particle and the side wall of the channel.

Advantageously, the structure of the device is simpler to produce since the substrate channel has a size in the order of tens of nanometres.

### LIST OF FIGURES

Further characteristics and advantages of the present invention will become more apparent from the approximate and thus non-limiting description of a preferred, but not exclusive, embodiment of a device and a method for detecting molecules, as illustrated in the accompanying drawings, in which:

FIG. 1 is a front sectional view of the device according to the present invention,

FIG. 2 is a front sectional view of the device with a support membrane and gel layer,

FIG. 3 is a top view of a pore which has surface irregularities.

### DETAILED DESCRIPTION

With reference to the accompanying drawings, 1 indicates a device for detecting molecules 100. The molecules 100 to be detected are, for example, proteins.

Such a device 1 comprises a substrate 2 with a first face 21 and a second face 22, opposite each other.

The substrate 2 comprises at least one through channel 3 having a first opening 31 at the first face 21 and a second opening 32 at the second face 22. Such channel 3 is delimited by a side wall 33, which extends between the first and the second face 21, 22. Preferably, the channel 3 has a main extension direction X which is orthogonal with respect to the first and the second face 21, 22. In the preferred embodiment, such a substrate 2 having a channel is a nanopore.

The device 1 comprises a receptacle 4 configured to contain a saline solution 41 and the molecule 100 to be detected. Immersed within such a receptacle 4 is the substrate 2 to be detected, which can be inserted in the saline solution 41 during use.

It should further be noted that the device 1 comprises a light source 5 configured to illuminate the channel 3 with a monochromatic electromagnetic radiation 51 such as to generate a hotspot within the channel 3. Preferably, such a light source 5 is a laser. Still preferably, the monochromatic electromagnetic radiation 51 has a wavelength in the visible or near-infrared spectrum. Preferably, the wavelength of the monochromatic electromagnetic radiation 51 is comprised between 400 and 800 nm.

The device 1 comprises a generator 23 configured to generate a potential difference between the first face 21 and the second face 22 of the substrate 2 such that, in use, the molecule 100 to be detected is moved within the channel 3. In detail, the potential difference is configured to move molecule 100 from the first opening 31 towards the second opening 32 by means of electrophoresis.

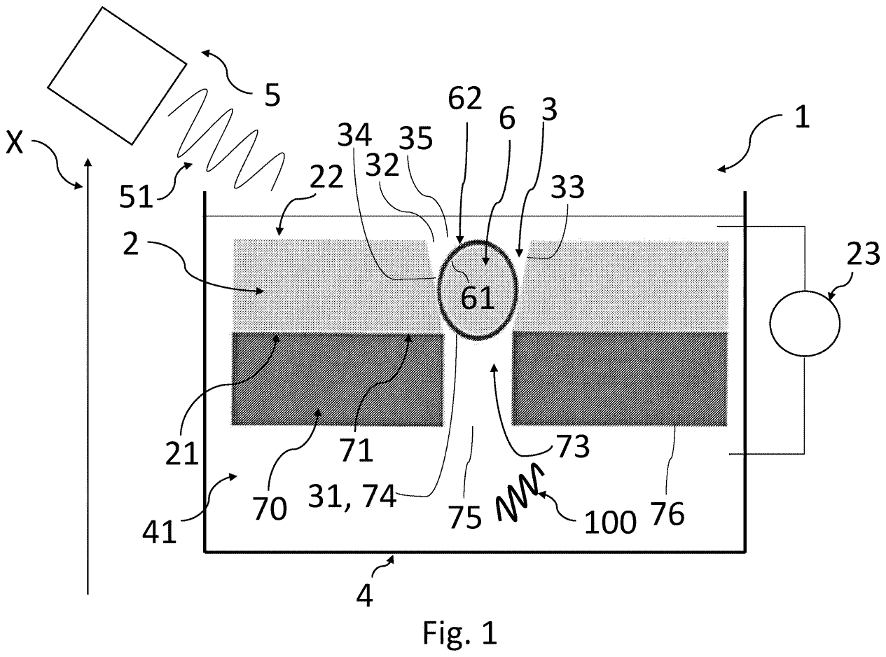

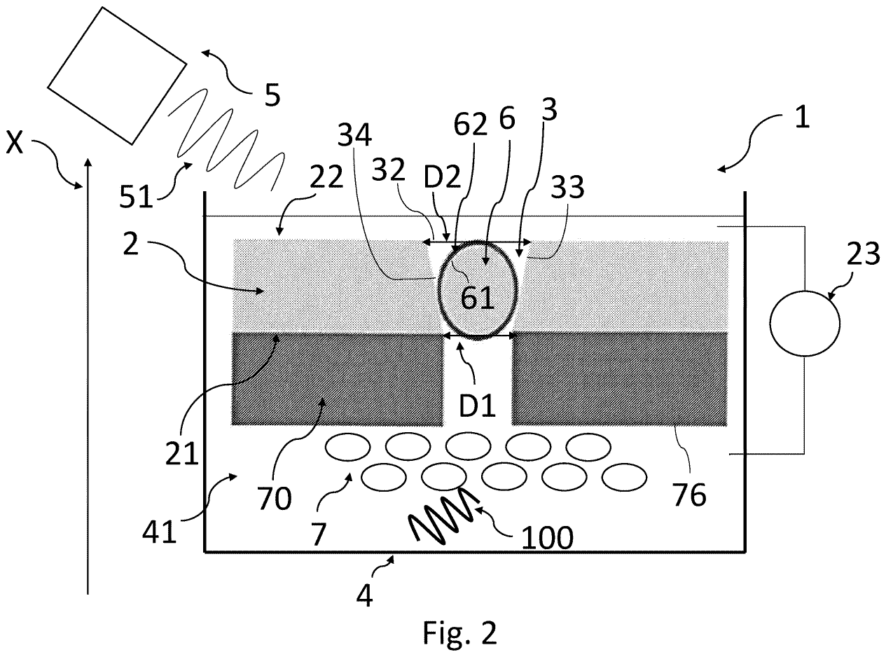

It should be noted that the device 1 comprises a particle 6 at least in part inserted in the channel 3. In more detail, the particle 6 is configured to at least partially obstruct the channel 3, as shown in FIG. 1 or 2.

It should be noted that the hotspot which can be generated is located between the particle 6 and the side wall 33 of the channel 3. Exciting the channel 3 with a monochromatic radiation 51, optical energy is concentrated in the channel 3, between the side wall 33 and the particle 6. Thanks to the partial obstruction of the channel 3 by the particle 6, an increase in the electric field strength between the particle 6 and the side wall 33 with respect to the electric field strength generated in the channel 3 itself can be detected. Advantageously, increasing the field strength improves the detection resolution.

In detail, there is a flow volume 34 between the particle 6 and the side wall 33 such as to allow the flow of the molecule 100 between the particle 6 and the side wall 33. In detail, the generable hotspot is positioned at least in part at the flow volume 34 between the particle 6 and the side wall 33. It is thereby possible to stimulate the molecule 100 to be detected. The molecule 100 is configured to emit a detection signal when it crosses the hotspot.

It should be noted that at least one among the particle 6 and the side wall 33 undergoes a surface treatment, known to the person skilled in the art. Preferably, the surface treatment comprises the deposition of aluminium oxide or organic molecules which in saline solution have charges of the same sign as the molecule to be detected. For example, organic molecules have a negative charge in saline solution when the molecule 100 is a DNA molecule. Preferably, the surface treatment prevents the molecule 100 from adhering to the side wall 33 of the channel 3 or the particle 6. Even more preferably, the surface treatment is configured to repel the molecule 100 from the side wall 33 or from the particle 6. In the preferred embodiment, both the particle 6 and the channel 33 undergo such a surface treatment.

Advantageously, the surface treatment and potential difference improve the flow of the molecule 100, preventing the molecule 100 from being blocked between the particle 6 and the side wall 33.

In an embodiment, the device 1 comprises a gel layer 7 positioned at the first opening 31 of the channel 3. Such a gel layer 7 is configured to be immersed in the saline solution 41 within the receptacle 4 and to be crossed by the molecule 100 for detection when said molecule 100 enters the channel 3. In an embodiment, the gel layer 7 is for example agarose.

In detail, when the molecule 100 crosses the gel layer 7, such a molecule 100 is slowed down, unfolded and elongated as it enters the channel 3.

Advantageously, the gel layer 7 improves the detection of the molecule 100, as detection signals emitted by each portion of molecule 100 can be detected more accurately. In more detail, by unfolding and elongating the molecule 100, each detection signal emitted by the molecule 100 at a given instant in time is that related to a single portion of molecule 100. Furthermore, by slowing down the flow of the molecule 100, it is possible to stimulate the molecule 100 for longer, obtaining a more accurate detection signal. For example, if the molecule 100 to be detected is a protein, the detection signals emitted by different amino acids can be detected more accurately.

In a further embodiment, electrosmosis or thermophoresis techniques can be used to control the flow of the molecule 100. As known to the person skilled in the art, for the thermophoresis process, a temperature gradient is applied, preferably between the first face 21 and the second face 22 of the substrate 2.

The first opening 31 of the channel 3 has a first characteristic dimension D1.

In the context of the present invention, for characteristic dimension is meant a dimension adapted to define the scale of the physical reference system and is defined as a function of the specific geometry considered. As an example, in a plane geometry, the characteristic dimension can be defined by the respective length, while in a tubular geometry it can be defined by the diameter.

Preferably, the first characteristic dimension D1 is substantially a diameter, for example as schematically shown in FIG. 2.

It should be noted that, in the device 1, the first opening 31 has surface irregularities, e.g., surface roughness or grain boundaries, at a first perimeter, viz. on the side wall 33 of the channel 3.

Furthermore, the second opening 32 of the channel 3 has a second characteristic dimension D2, which is greater than the first characteristic dimension D1. Preferably, the second characteristic dimension D2 is a diameter, schematically shown in FIG. 2.

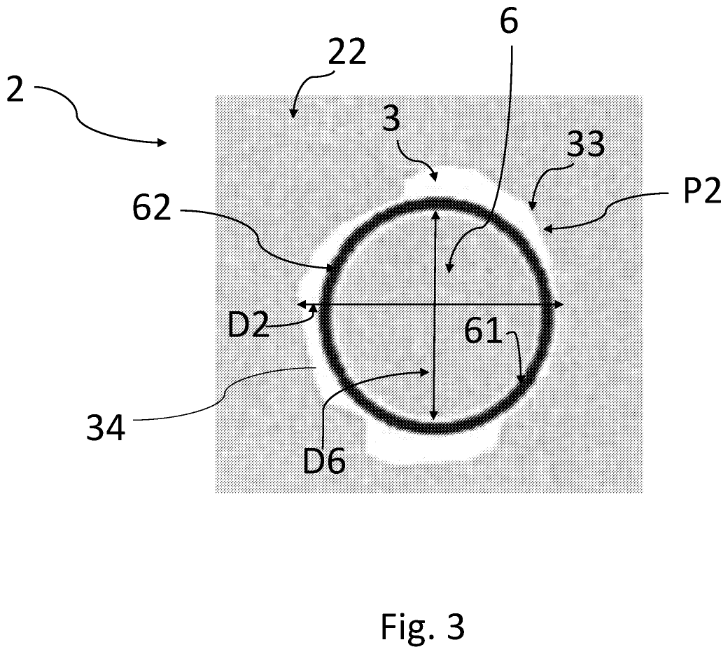

Similarly to the first opening 31, the second opening 32 also has surface irregularities, e.g., surface roughness or grain boundaries, at a second perimeter P2, viz. on the side wall 33 of the channel 3, as schematically shown in FIG. 3.

In the preferred embodiment, the particle 6 is spherical in shape. It should also be noted that the particle 6 has a particle characteristic dimension D6 comprised between the first and the second characteristic dimensions D1, D2. Preferably, the particle characteristic dimension D6 is a diameter of the particle 6, schematically shown in FIG. 3. The particle 6 is also at least partially inserted in the channel 3 at the second opening 32.

It should be noted that the surface irregularities which may be present in the first and in the second opening 31, 32 can be controlled to obtain an optimal first and second characteristic dimension D1, D2. In other words, the surface irregularities of the second opening 32 can be adjusted to optimise, preferably reduce, the difference between the second characteristic dimension D2 and the particle characteristic dimension D6. Thereby, it is possible to strengthen the generable hotspot.

It should be noted that the particle 6 is inserted in the channel 3 by methods known to the person skilled in the art, e.g. electrophoresis, functionalisation or drop casting.

Furthermore, since the particle 6 has a characteristic dimension D6 greater than the second characteristic dimension D2, the particle 6 is trapped within the channel 3, as shown in FIGS. 1 and 4. Once within the channel 3, the particle 6 binds to the side wall 33 by means of non-specific adhesion or chemical functionalisation.

In an embodiment, the light source 5 is configured to generate linearly polarised monochromatic light radiation 51. In such an embodiment, the generable hotspot is not uniformly distributed between the particle 6 and the side wall 33.

In detail, in such embodiment, the generable hotspot has at least a first zone with maximum strength and at least a second zone with minimum strength. It should be noted that the position of the first and the second zone depends on the polarisation direction of the monochromatic light radiation 51. Such a polarisation direction is presettable by a user.

In the preferred embodiment, the light source 5 is configured to generate circularly polarised monochromatic light radiation 51.

In the context of the present invention, circularly polarised monochromatic light radiation 51 is meant as monochromatic light radiation having angular momentum.

In the preferred embodiment, the generable hotspot is uniformly distributed between the particle 6 and the side wall 33.

Circular polarisation of the monochromatic radiation 51 is preferred with respect to linear polarisation because the molecule 100 can be detected crossing the channel 3 at any point between the particle 6 and the side wall 33. In fact, since the electric field strength in the generable hotspot in such preferred embodiment is homogeneous at all points, each detection signal emitted by the molecule 100 has the same strength.

Advantageously, in such a preferred embodiment, the detection is easily reproducible.

It should be noted that, under the same experimental conditions, the strength of the hotspot generable by the circularly polarised monochromatic light radiation 51 is less than the maximum electric field strength measured in the first zone and greater than the minimum electric field strength measured in the second zone of the hotspot generable by the linearly polarised monochromatic light radiation 51.

It should be noted that the wavelength of the monochromatic light radiation 51 must be selected by a user depending on parameters such as the geometry of the channel 3 and the materials used, so as to generate a hotspot. For example, a substrate 2 made of silver requires a shorter wavelength of monochromatic light radiation 51 with respect to a substrate 2 made of gold for the same channel 3 geometry.

In a preferred embodiment of the device 1, at least one among the substrate 2 and the particle 6 is made of plasmonic material. In the context of the present invention, a plasmonic material is meant as a conducting material with a plasma frequency greater than the frequency of the radiation used to generate the hotspot. Typical examples of plasmonic materials in the visible spectrum are the noble metals. In detail, such an embodiment of the device 1 is configured to optically detect the molecule 100, e.g. by means of Raman spectroscopy.

For example, the substrate 2 and the particle 6 can both be made of plasmonic materials.

In an embodiment in which the substrate 2 is made of a plasmonic material and the particle 6 is made of a non-plasmonic material, the particle 6 is configured to partially occlude the channel 3.

In the context of the present invention, non-plasmonic materials are meant as insulating materials, e.g., silicon oxide, or polymers, e.g., polystyrene, or low doping semiconductors, e.g., silicon.

It should be noted that the particle 6 comprises an outer surface 61 configured to be covered by at least one insulating layer 62 so as to generate a controlled separation between the side wall 33 and the particle 6. It is known that when such a separation is only a few atomic layers, e.g., comprised between 1 and 3 nm, the hotspot strength is increased. Preferably the insulating layer 62 is for example silicon oxide, titanium oxide or alumina or another insulating material with a low Raman emissivity. In an alternative embodiment, the side wall 33 of the channel 3 is coated with the insulating layer 62.

It should be noted that if the difference between the second characteristic dimension D2 and the particle characteristic dimension D6 is smaller than a few atomic layers, e.g., smaller than a range comprised between 1 and 3 nm, the electric field strength of the generable hotspot decreases due to electron tunnelling of the plasmonic material or short-circuit effects. By lining the particle 6 or the side wall 33 with the insulating layer 62, it is possible to limit, preferably eliminate, tunnelling or short-circuit effects, restoring the electric field strength of the hotspot, as a capacitive behaviour is restored between the particle 6 and the side wall 33.

In other words, the particle 6 and the side wall 33 behave like a capacitor. When the separation between the particle 6 and the side wall 33 is less than a few atomic layers, i.e., less than a range comprised between 1 and 3 nm, the capacitor behaves as if short-circuited. Placing an insulating layer 62 partly restores the operation of the capacitor, but the insulating layer 62 must be thin in order to keep the capacitance of the capacitor high. The realistic minimum insulating layer 62 is about 1 nm, then tunnelling phenomena occur which short-circuit the capacitor.

It should be noted that the generator 23 is configured to generate a minimum potential difference, between the first and the second face 21, 22, such that the molecule 100 to be detected in the saline solution 41 can flow between the first and the second opening 32, 33.

In fact, such currents flow in the opposite direction with respect to the molecule 100. Advantageously, by adjusting the potential difference, it is possible to adjust the translocation time of the molecule 100.

It should be noted that, in a preferred embodiment, the device 1 comprises a support membrane 70 on which the substrate 2 is deposited. In detail, the substrate 2 is in contact with a first support face 71 by means of the first face 21. Furthermore, in such a preferred embodiment, the support membrane 70 has a further channel 73 which is continuous with respect to the channel 3, along the main extension direction X.

It should be noted that such a further channel 73 has a first support opening 74, coinciding with the first opening 31, and a second support opening 75, spaced from the first support opening 74 along the main extension direction X.

In such a preferred embodiment, the aerogel layer 7 is positioned at a second support face 76, opposite the first support face 71.

Furthermore, in such an embodiment, the potential difference is applied between the second support face 76 of the support membrane 70 and the second face 22 of the substrate 2. Thereby, the molecule 100 is configured to flow between the second support opening 75 and the second opening 32 of the channel 3.

In more detail, such a support membrane 70 is configured to be immersed in the saline solution 41 in use.

Furthermore, the support membrane is made of, for example, silicon nitride.

It should be noted that the substrate 2 which comprises the first face 21 and the second face 22. The first and the second face 21, 22 are opposite each other and are spaced by a thickness comprised between 5 and 500 nanometres. The through channel 3 of the substrate 2 has a first opening 31 at the first face 21 with a first characteristic dimension D1 comprised between 5 and 200 nanometres, and a second opening 32 at the second face 22 with a second characteristic dimension D2 comprised between 5 and 200 nanometres. Furthermore, the channel 3 is delimited by a side wall 33 which extends between the first and the second face 21, 22. A particle 6 is configured to be at least in part inserted in the channel 3 at the second opening 32 and has a particle characteristic dimension D6 comprised between 5 and 200 nanometres.

Having described the device 1, its operating method, also an object of the present invention, will now be illustrated.

The method for detecting molecules 100 by means of the device 1 comprises a step of inserting the saline solution 41 and the molecule 100 to be detected in the receptacle 4.

Next, the method includes a step of inserting the substrate 2 in the receptacle 4 to immerse it in the saline solution 41.

The method includes a further step of illuminating the at least one channel 3 with the light source 5 so as to generate the hotspot between the particle 6 and the side wall 33 of the channel 3.

Furthermore, the method includes a step of generating the potential difference between the first and the second face 21, 22 with the generator 23 of the substrate 2 to make the molecule 100 flow between the particle 6 and the side wall 33 at the hotspot.

The method includes a further step of detecting the detection signal generated by the molecule 100 located at the hotspot.

If the detection signal is an optical signal, such an optical signal can be detected with instruments adapted to detect the Raman spectrum. In an alternative embodiment, other optical signals such as fluorescence or luminescence can be detected.

In a further embodiment, if the detection signal is of the electrical type, such a signal is a current with low intensity.

In a further embodiment, it is possible to detect both optical and electrical signals within the same experiment.

The Raman spectrum is obtained using a camera provided with a plurality of spectral points or pixels. In the Raman spectrum, a number of spectral bands can be identified, which, specifically, can discriminate different amino acids and the noise floor. It is also possible to limit the measurement to selected spectral bands. For each band, the total strength of the emitted signal, corresponding to the area subtended by the curve, can be measured.

Advantageously, to identify a protein, it is not necessary to measure the sequence of all the amino acids. In fact, the sequence of two or three amino acids, with respect to the total of twenty, is sufficient to identify most of the known proteins. One or more spectral points can be measured for each spectral band. In the case of a single spectral point, the strength of the spectrum in that region is obtained.

Furthermore, by means of the use of advanced data analysis and artificial intelligence techniques, it is possible to identify which and how many amino acids are required for a correct identification of the entire proteome or a portion thereof. Advantageously, by means of such advanced data analysis techniques, it is possible to identify the most significant portions of the spectrum adapted to discriminate one or more amino acids.

Consequently, for the identification of proteins by means of Raman spectroscopy and plasmonic nanopores, it is sufficient to develop a system for measuring specific spectral areas without necessarily measuring the entire Raman spectrum. In particular, it may be advantageous to measure the strength of two spectral areas corresponding to the region from 600 to 1600 cm⁻¹ and the region from 2800 to 3400 cm⁻¹, or the ratio of the strengths thereof. In the region between about 1800 cm⁻¹ and 2800 cm⁻¹, the biological molecules do not emit Raman signals.

The project from which the present patent application is derived received funding from the European Union's research and innovation programme Horizon 2020 GA 964363.

1. Device for detecting molecules comprising:
a substrate comprising a first face and a second face, opposite each other, the substrate comprising at least one through channel having a first opening at the first face and a second opening at the second face, the channel being delimited by a side wall extending between the first and the second face,
a receptacle configured to contain a saline solution and the molecule to be detected, the substrate being insertable in the receptacle to be immersed in the saline solution being used,
a light source configured to illuminate the channel with a monochromatic electromagnetic radiation such that a hotspot is generated within the channel,
a generator configured to generate a potential difference between the first face and the second face of the substrate such that during use the molecule to be detected in the channel is moved between the first and the second opening,
wherein
the device comprises a particle at least in part inserted in the channel,
the generable hotspot is located between the particle and the side wall of the channel,
the particle and the side wall are spaced so that the molecule to be detected, which emits an optically or electrically detectable detection signal when crossing the hotspot, flows between the particle and the side wall at the hotspot.

2. Device according to claim 1 comprising a gel layer positioned at the first opening of the channel, configured to be immersed in the saline solution within the receptacle and to be crossed by the molecule for detection when said molecule enters the channel.

3. Device according to claim 1, wherein the light source is configured to generate a linearly polarised monochromatic light radiation.

4. Device according to claim 1, wherein the light source is configured to generate a circularly polarised monochromatic light radiation.

5. Device according to claim 1, wherein at least one among the substrate and the particle is made of plasmonic material.

6. Device according to claim 5, wherein the particle comprises an outer surface coated with at least one layer of insulating material when the distance between the side wall and the outer surface at a plane defined by the second opening is less than 1 nanometre.

7. Device according to claim 1, wherein the generator is configured to generate a minimum potential difference that allows the molecule to be detected in the saline solution to flow between the first and the second opening.

8. Device according to claim 1, wherein the first opening of the channel has a first characteristic dimension, the second opening of the channel having a second characteristic dimension, which is greater than the first characteristic dimension, the particle having a particle characteristic dimension comprised between the first and the second characteristic dimension and being at least partially inserted in the channel at the second opening.

9. Device according to claim 8, wherein the first face and the second face are spaced by a thickness comprised between 5 and 500 nm, the first characteristic dimension of the first opening being comprised between 5 and 200 nm, the second characteristic dimension of the second opening being comprised between 5 and 200 nm, the particle characteristic dimension of the particle being comprised between 5 and 200 nanometres.

10. Method for detecting molecules by means of a device according to claim 1, comprising the steps of:
inserting in the receptacle the saline solution and the molecule to be detected, which emits an optically or electrically detectable detection signal when it crosses the hotspot,
inserting the substrate in the receptacle by immersing it in the saline solution,
generating a hotspot between the particle and the side wall of the channel by illuminating the at least one channel with the light source,
generating the potential difference between the first and the second face of the substrate with the generator to make the molecule flow between the particle and the side wall at the hotspot,
detecting the detection signal generated by the molecule located at the generated hotspot.
